(19) United States
(12) Patent Application Publication
(10) Pub. No.: US 2004/0043946 A1
(43) Pub. Date: Mar. 4, 2004

(54) Topical formulations for treatment of skin disorders

(76) Inventor: Karl F. Popp, Schodack Landing, NY (US)

Correspondence Address: NATH & ASSOCIATES PLLC, Sixth Floor, 1030 Fifteenth Street, N.W., Washington, DC, 20005-1503, US

(21) Appl. No.: 10/617,191

(22) Filed: Jul. 11, 2003

Related U.S. Application Data
(60) Provisional application No. 60/407,285, filed on Sep. 3, 2002.

(51) Int. Cl. A61K 31/704; A61K 31/192

(52) U.S. Cl. 514/35; 514/568

(57) ABSTRACT

Topical compositions and methods of using same for treating skin disorders or conditions comprising a storage-stable mixture of a benzoyl peroxide dispersion, clindamycin or pharmaceutically acceptable salts or esters thereof, and a pharmaceutically acceptable carrier.

### FIELD OF THE INVENTION

The present invention relates to a topical composition and methods of using same for treating skin disorders or conditions comprising a storage-stable mixture of a benzoyl peroxide dispersion, clindamycin or pharmaceutically acceptable salts or esters thereof, and a pharmaceutically acceptable carrier.

### BACKGROUND OF THE INVENTION

Skin disorders involving the sebaceous glands and follicles in humans include conditions such as acne and rosacea, as well as other noninfectious dermatological diseases involving microorganisms. Such disorders are often marked by inflammation.

Acne is a common skin disorder characterized by blackheads, whiteheads, papules, pustules, cysts, and various sized nodules and scars which, in the inflammatory state of the disorder, are contaminated with bacteria such as Propionibacterium acnes. The disorder affects skin areas where the sebaceous glands are most active, and bacterial infection can occur in the sebaceous follicles.

In the past, these dermatological disorders have been treated with oral and/or topical antibacterial agents. The oral antibiotics used include tetracycline, erythromycin, and minocycline. The topical compositions used have separately contained the antibiotics tetracycline, erythromycin, and clindamycin, as well as benzoyl peroxide, which exerts its antibacterial action via its potent oxidizing properties. However, the strong oxidizing properties of peroxide result in unstable compositions. Benzoyl peroxide also can act as a sebosuppressant, an irritant, and a comedolytic agent.

One currently available product, Cleocin T® brand clindamycin phosphate topical solution by Pharmacia & Upjohn Company of Kalamzoo, Mich., is a topical solution containing 1% of clindamycin phosphate. Cleocin T®, however, has several drawbacks. For one, the formulation contains 50% isopropyl alcohol and water. This formulation often proves to be excessively drying and irritating to the skin. Second, the composition as dispensed by the pharmacist lacks the stability necessary for extended storage at room temperature.

Topical compositions combining at least two active antibacterial agents have been proposed as a treatment to these disorders. These compositions typically require compounding by the pharmacist and must be refrigerated. After three months of refrigeration, the compositions lose potency and effectiveness and must be replaced with a new batch.

For example, a currently available combination product is Benzamycin® brand topical gel (Dermik Laboratories, Berwyn, Pa.) which contains 3% of erythromycin and 5% of benzoyl peroxide. Benzamycin®, however, has several drawbacks. First, the product is supplied to pharmacies as a benzoyl peroxide gel in a first container and erythromycin powder in a second container. The product thus requires compounding by the pharmacist, who must (1) dissolve the erythromycin in alcohol, (2) add the erythromycin solution to the gel, and (3) stir until homogeneous in appearance. Second, the alcohol present in the composition as dispensed amounts to 16% of the total composition, which has proven to be excessively drying and irritating to the skin, particularly in combination with the benzoyl peroxide. Third, the composition as dispensed by the pharmacist (i.e., after reconstitution or compounding) lacks the stability necessary for extended storage at room temperature. The combination product can be stored under refrigeration for up to three (3) months.

Similarly, the currently available combination product BenzaClin® is a topical gel containing 1% of clindamycin and 5% of benzoyl peroxide. BenzaClin®, however, also has several drawbacks. For example, the product must be compounded by a pharmacist since it is supplied to pharmacies as a benzoyl peroxide gel in a first container and clindamycin powder in a second container. Accordingly, it lacks the stability necessary for extended storage at room temperature since the combined product can only be stored for up to two (2) months. By requiring compounding by pharmacists, it also has variability/impurity problems, which are the result of the drug forming partially dissolved or undissolved aggregates. This causes some patients to report that the product sometimes feels “gritty” when applied to the skin, further exacerbating the inflammation and irritation problem due to skin abrasion. Lastly, this composition must be topically applied at least twice a day to be effective in accordance with label directions.

Other efforts at improving the stability of combination products in particular have relied on the use of novel packaging that keeps the active agents separated to maintain stability until the time of use. However, compounding is still necessary at the time of dispensing, and stability remains a problem because the product must be used immediately upon being prepared.

Another known topical composition for the treatment of acne is described in U.S. Pat. No. 6,117,843, the entire contents of which are herein incorporated by reference. This patent describes topical therapeutic compositions for the treatment of acne containing a combination of benzoyl peroxide and clindamycin. The clindamycin used in the disclosed compositions has a pre-combination pH of 5.9 to 6.9. Additionally, the disclosed compositions must be administered twice a day to be effective for the treatment of acne.

The presently known compositions for the treatment of acne are formulated for administration to patients twice per day and it has been reported that patient compliance with compositions that must be administered twice per day tends to be irregular, especially among teenagers who are the primary sufferers of acne.

Lastly, the current treatment options pose a significant risk of adverse side effects. Clindamycin, which is well absorbed through the skin, has been associated with colitis, diarrhea, and bloody diarrhea. Severe colitis may result in death. Accordingly, there is a need to reduce the potential side effects of these prior compositions by reducing the number of required daily exposures to them.

For these reasons, it would be desirable to provide improved compositions and methods for formulating compositions for the treatment of acne. In particular, it would be desirable to provide products combining the activity of an antibiotic compound, such as clindamycin, with the activity of benzoyl peroxide, with few or none of the disadvantages described above. Such compositions should overcome the formulation and stability problems which have been associated with the prior compositions, and provide improved compositions which are less irritating, easy to formulate, have a smooth consistency after formulation, are adequately stable, and have a sufficiently long storage life with or without refrigeration.

Accordingly, there remains a need for a topical composition for the treatment of skin disorders that is storage-stable for an extended period of time, easy to formulate, substantially uniform, and has a sufficiently long storage life with or without refrigeration.

### SUMMARY OF THE INVENTION

The present inventive subject matter relates to a topical composition for treating a skin disorder or condition, which comprises:

a storage-stable mixture of a benzoyl peroxide dispersion, clindamycin or a pharmaceutically acceptable salt or ester thereof, and a pharmaceutically acceptable carrier,

wherein the composition has a final pH of about 4.5 to about 5, and wherein the composition has a viscosity lower than the viscosity of the benzoyl peroxide dispersion before mixing.

In a preferred embodiment, the present inventive subject matter also relates to a method for treating a skin disorder or condition in a patient comprising topically administering to a patient in need thereof a topical composition in an amount effective to treat said skin disorder, wherein said composition comprises:

a storage-stable mixture of a benzoyl peroxide dispersion, clindamycin or a pharmaceutically acceptable salt or ester thereof, and a pharmaceutically acceptable carrier,

wherein the composition has a final pH of about 4.5 to about 5, and wherein the composition has a viscosity lower than the viscosity of the benzoyl peroxide dispersion before mixing.

In another preferred embodiment, the present inventive subject matter relates to a process for preparing a storage-stable topical composition for treating for a skin disorder or condition, which comprises the steps of:

a) forming at a temperature of about 15 to about 25 ° C. a benzoyl peroxide intermediate dispersion having between about 5.9% and about 7.2% benzoyl peroxide and having a viscosity of about 60,000 to about 250,000 centipoises;

b) forming at a temperature of about 15 to about 25 ° C. a clindamycin intermediate solution sufficient to yield a composition which contains between about 0.5% and about 1.5% by weight clindamycin active in the final product; and

c) mixing said benzoyl peroxide intermediate dispersion and said clindamycin intermediate solution under conditions sufficient to yield a benzoyl peroxide and clindamycin mixture having final pH of between about 4.5 to about 5.0,

wherein said mixture has a viscosity of about 50,000 to about 200,000 centipoises, and wherein said composition comprises sufficient inactive ingredients to provide storage stability and effectiveness for a treatment period.

### DETAILED DESCRIPTION OF THE INVENTION

### Definitions

The term “sensitivity” refers to the degree of skin irritation or skin inflammation, as exemplified by parameters in suitable assays for measuring sensitivity, inflammation, irritation, and the like. One such assay is the Jordan-King assay.

The term “commercial purposes” refers to any purposes requiring any length of time or storage condition in accordance with FDA rules or regulations, including shipping time, storage, distribution, and refrigeration.

The term “acne” means a common inflammatory disease of the pilosebaceous glands characterized by comedones, papules, pustules, inflamed nodules, superficial pus-filled cysts, and (in extreme cases) canalizing and deep, inflamed, sometimes purulent sacs. Types of acne within the scope of the present inventive subject matter include acne vulgaris or topical acne. “Acne” is caused by an interaction among hormones, keratin, sebum, and bacteria. One common bacterial causative agent is Propionibacterium acnes.

### Compositions

The present inventive subject matter relates to a topical composition for treating a skin disorder or condition, which comprises:

a storage-stable mixture of a benzoyl peroxide dispersion, clindamycin or a pharmaceutically acceptable salt or ester thereof, and a pharmaceutically acceptable carrier,

wherein the composition has a final pH of about 4.5 to about 5, and wherein the composition has a viscosity lower than the viscosity of the benzoyl peroxide dispersion before mixing.

The benzoyl peroxide component of the present inventive compositions is introduced as a dispersion. The benzoyl peroxide is pharmaceutical grade. The benzoyl peroxide in the dispersion may be in the form of a slurry of a finely divided powder, or in the form of a hydrous granular material which may have its particle size reduced accordingly during processing according to the present inventive subject matter. Preparation of suitable benzoyl peroxide constituents is well described in the medical and patent literature.

The benzoyl peroxide component of the present inventive compositions is generally present at an amount of between about 1.0% to about 20.0% by weight of the total composition of benzoyl peroxide. In a preferred embodiment, the compositions contain from about 2.25% to about 12.5% by weight of the total composition of benzoyl peroxide. In a particularly preferred embodiment, the present compositions contain about 11% by weight of benzoyl peroxide. The present inventive compositions are unique in that they can be produced having a standard deviation of benzoyl peroxide present within ±0.07.

Additionally, the present inventive compositions effectively maintain a benzoyl peroxide composition having not more than 0.01% by weight of benzoyl peroxide impurities.

In a preferred embodiment, the benzoyl peroxide used in the present inventive compositions is about 65% to about 80% pure, the remainder being purified water.

Prior to mixing, the benzoyl peroxide dispersion has a preferred viscosity of about 60,000 to about 250,000 centipoises. In a particularly preferred embodiment, the benzoyl peroxide dispersion has a viscosity of about 110,000 to about 220,000 centipoises.

The clindamycin component of the present inventive compositions is preferably a pharmaceutical grade salt or ester of clindamycin. Pharmaceutically acceptable salts, esters, or solvates of clindamycin refer to those which possess the desired pharmacological activity and which are neither biologically nor otherwise undesirable. The salts, esters, or solvates can be formed with inorganic or organic acids such as acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, gluconate, glycerophosphate, hemisulfate, heptanoate, hexanoate, hydrochloride, hydrobromide, hydroiodide, 2-hydroxyethanesulfonate, lactate, maleate, methanesulfonate, naphthylate, 2-naphthalenesulfonate, nicotinate, oxalate, sulfate, thiocyanate, tosylate and undecanoate.

Base salts, esters, or solvates useful herein include ammonium salts, alkali metal salts such as lithium, sodium and potassium salts, alkaline earth metal salts such as calcium and magnesium salts, salt with organic bases such as dicyclohexylamine salts, N-methyl D-glucamine, and salts with amino acids such as arginine, lysine, and so forth. Also the basic nitrogen-containing groups can be quarternized with such agents as: 1) lower alkyl halides, such as methyl, ethyl, propyl, and butyl chloride, bromides and iodides; 2) dialkyl sulfates like dimethyl, diethyl, dibutyl and diamyl sulfates; 3) long chain alkyls such as decyl, lauryl, myristyl and stearyl substituted with one or more halide such as chloride, bromide and iodide; and 4) aryl or arylalkyl halides like benzyl and phenethyl bromide and others.

Clindamycin phosphate (ester) and clindamycin hydrochloride (salt) are preferred pharmaceutically acceptable salts and esters of clindamycin which can be used in the present composition due to their compatibility with gelling agents and extensive history of topical use.

The clindamycin component of the present inventive compositions is generally present at an amount of from about 0.90% to about 2.5% by weight of the total composition. In a preferred embodiment, the present inventive compositions contain between about 0.5% and about 1.5% by weight of the composition clindamycin. In a particularly preferred embodiment, the present inventive compositions contain about 1.2% by weight clindamycin. The present inventive compositions are unique in that they can be produced having a standard deviation of clindamycin present within ±0.015.

Additionally, the present inventive compositions effectively maintain a clindamycin composition having not more than 0.02% by weight of clindamycin degradates.

In the final composition, the ratio of benzoyl peroxide to clindamycin may be from about 1.8:1 to 12:1. Particularly preferred are compositions wherein the ratio of benzoyl peroxide to clindamycin is from about 4:1 to about 5:1. Further, the final compositions preferably have a final viscosity of about 50,000 to about 200,000 centipoises. In a particularly preferred embodiment, the final compositions have a final viscosity of about 100,000 to about 200,000 centipoises. This final viscosity that is lower than the viscosity of the benzoyl peroxide dispersion demonstrates that the present inventive compositions are easier to mix together, contain less degradates, and have a greater degree of uniformity than those compositions previously known in the art.

In a preferred embodiment, the final compositions exhibit a final pH of about 4.5 to about 5. In a particularly preferred embodiment, the final compositions exhibit a final pH of about 4.6 to about 4.8. This narrowly tailored pH is in part responsible for the advanced storage stability of the present inventive compositions in comparison to those previously known in the art.

The present inventive compositions do not require compounding at the time of dispensing and maintain stability indefinitely depending on the storage temperature, despite the relative incompatibility of benzoyl peroxide and clindamycin. This represents a distinct advantage over the formulations presently known in the art.

The present inventive compositions may be formulated for either once-per-day or twice-per-day administration. In a preferred embodiment, the once-per-day administration is in the morning to increase compliance and to account for skin conditions most favorable to reducing inflammation. An additional advantage of administration in the morning is the minimization of the risk of bleaching fabrics occasionally seen when a patient puts a benzoyl peroxide product on their face at night and the medication comes in contact with a “colored” pillow case or sheet, etc. resulting in a white spot on the fabric.

The benzoyl peroxide dispersion, as well as the final composition, may take the form of a gel, cream, lotion, suspension, emulsion, ointment, or foam. Other cosmetic treatment compositions known to those skilled in the art, including liquids and balms, are additionally contemplated as falling within the scope of the present inventive subject matter.

Emulsions, such as oil-in-water or water-in-oil systems, as well as a base (vehicle or carrier) for the topical formulation is selected to provide effectiveness of the active ingredient and/or avoid allergic and irritating reactions (e.g., contact dermatitis) caused by ingredients of the base or by the active ingredients.

Creams useful in the present inventive compositions may also be semisolid emulsions of oil and water; are easily applied and vanish when rubbed into the skin.

Lotions useful in the present inventive compositions include older definitions such as suspensions of powdered material (e.g., calamine) in a water or alcohol base, as well as modern lotions (e.g., some corticosteroids) such as water-based emulsions. Convenient to apply, lotions are also cool and help to dry acute inflammatory and exudative lesions.

Suitable lotions or creams containing the active compound may be suspended or dissolved in, for example, a mixture of one or more of the following: mineral oil, sorbitan monostearate, polysorbate 60, cetyl ester wax, de minimis cetearyl alcohol, de minimis 2-octyldodecanol, de minimis benzyl alcohol and water.

Ointments which are useful are oleaginous and contain little if any water; feel greasy but are generally well tolerated; best used to lubricate, especially if applied over hydrated skin; they are preferred for lesions with thick crusts, lichenification, or heaped-up scales and may be less irritating than cream for some eroded or open lesions (e.g., stasis ulcers). Drugs in ointments are often more potent than in creams.

The compounds can be formulated into suitable ointments containing the compounds suspended or dissolved in, for example, mixtures with one or more of the following: mineral oil, liquid petrolatum, white petrolatum, propylene glycol, polyoxyethylene polyoxypropylene compound, emulsifying wax and water.

In severe cases, occlusive therapy may be useful where acne presents concurrently with other indications or conditions such as psoriasis, atopic dermatitis, lupus erythematosus, and chronic hand dermatitis. Covering the treated area with a nonporous occlusive dressing increases the absorption and effectiveness of topical corticosteroids. Usually, a polyethylene film (plastic household wrap) is applied overnight over cream or ointment, which tends to be less irritating than lotion for occlusive therapy. Plastic tapes may be impregnated with drug and is especially convenient for treating isolated or recalcitrant lesions; children and (less often) adults may experience pituitary and adrenal suppression after prolonged occlusive therapy over large areas.

Suitable gelling agents which may be useful in the present compositions include aqueous gelling agent, such as neutral, anionic, and cationic polymers, and mixtures thereof. Exemplary polymers which may be useful in the instant compositions include carboxy vinyl polymers, such as carboxypolymethylene. A preferred gelling agent is Carbopol® brand Carbopol polymer such as is available from Noveon Inc., Cleveland, Ohio. Suitable gelling agents include Carbopol polymers. Carbopol polymers are high molecular weight, crosslinked, acrylic acid-based polymers. Carbopol homopolymers are polymers of acrylic acid crosslinked with allyl sucrose or allylpentaerythritol. Carbopol copolymers are polymers of acrylic acid, modified by long chain (C10-C30) alkyl acrylates, and crosslinked with allyl-pentaerythritol.

Other suitable gelling agents include cellulosic polymers, such as gum arabic, gum tragacanth, locust bean gum, guar gum, xanthan gum, cellulose gum, methylcellulose, hydroxyethylcellulose, hydroxypropylcellulose, and hydroxypropylmethylcellulose.

Any non-toxic, inert and effective carrier may be used to formulate the present inventive compositions. Well-known carriers used to formulate other therapeutic compounds for administration to humans particularly will be useful in the compositions of the present invention. Pharmaceutically acceptable carriers, excipients and diluents in this regard are well known to those of skill, such as those described in The Merck Index, Thirteenth Edition, Budavari et al., Eds., Merck & Co., Inc., Rahway, N.J. (2001), which is incorporated by reference herein in its entirety. Examples of such useful pharmaceutically acceptable excipients, carriers and diluents include distilled water, physiological saline, Ringer's solution, dextrose solution, Hank's solution and DMSO, which are among those preferred for use in the present invention.

These additional components, as well as effective formulations and administration procedures are well known in the art and are described in standard textbooks, such as Goodman and Gillman's: The Pharmacological Bases of Therapeutics, 8th Ed., Gilman et al. Eds. Pergamon Press (1990) and Remington's Pharmaceutical Sciences, 17th Ed., Mack Publishing Co., Easton, Pa. (1990), both of which are incorporated by reference herein in their entirety.

Examples of preferred inactive ingredients that can be used according to the present inventive compositions include but are not limited to carbomer, disodium monolauryl sulfosuccinate, disodium EDTA, methyl paraben, poloxamer, glycerin, dimethicone, hydrated silica, sodium hydroxide, purified water, and mixtures thereof.

Other ingredients which may optionally be provided in the instant topical compositions include humectants, such as propylene glycol; solvents, such as alcohol (de minimis); and anti-microbial preservatives, such as methylparaben and propylparaben. The topical compositions may also include an organic or inorganic base, such as sodium hydroxide, which is used to adjust the pH of the initial components and the final product.

### Additional Active Ingredients

In addition to the benzoyl peroxide and clindamycin, the present inventive compositions may further contain other active ingredients readily known to those of skill in the art as useful in the topical treatment of skin disorders or conditions. Exemplary additional active ingredients include, but are not limited to, other macrolide antibiotics, bactericidal drugs, bacteriostatic drugs, cleansing agents, absorbents, anti-infective agents, anti-inflammatory agents, astringents (drying agents that precipitate protein and shrink and contract the skin), emollients (skin softeners), moisturizers, keratolytics (agents that soften, loosen, and facilitate exfoliation of the squamous cells of the epidermis), and mixtures thereof.

Exemplary macrolide antibiotics contemplated as within the scope of the invention include, but are not limited to, Azithromycin, Clarithromycin, Erythromycin, Lincomycin, and mixtures thereof. The macrolides are similar in structure and activity. All the macrolides are easily absorbed and all are primarily bacteriostatic and bind to the 50S subunit of the ribosome, thus inhibiting bacterial protein synthesis. These drugs are active against aerobic and anaerobic gram-positive cocci, with the exception of enterococci, and against gram-negative anaerobes and useful in the present invention.

Exemplary bactericidal drugs (i.e., they kill bacteria) contemplated as within the scope of the invention include, but are not limited to, Penicillins, cephalosporins, vancomycin, aminoglycosides, quinolones, and polymyxins.

Exemplary bacteriostatic drugs (i.e., they slow bacterial growth) contemplated as within the scope of the invention include, but are not limited to, erythromycin, tetracyclines, chloramphenicol, clindamycin, lincomycin, clarithromycin, azithromycin, and sulfonamides. However, it is well know that some bactericidal drugs may be bacteriostatic against certain microorganisms and vice versa. These drugs are well known in the art and may be found, for example, in The Merck Manual of Diagnosis and Therapy, 13^{th} edition, Section 13, Chapter 153 Anti-bacterial Drugs, 2001, incorporated herein by reference in its entirety.

Furthermore, the formulation may be used with adjunct therapies and treatments, such as pre-washing with common soaps, and mild detergents. However, selection is important when treating skin disorders such as acne since antibacterial soaps and abrasive soaps may increase irritation and make it difficult to use follicular drugs. Such follicular drugs may include topical antibiotics and antiseptics, as well as intralesional corticosteroids.

In superficial pustular acne, the topical benzoyl peroxide/clindamycin compositions may be used in combination with one of the follicular drugs.

Sunlight therapy can be useful in combination with the present inventive subject matter. Sunlight is known to cause mild dryness and slight scaling and is usually helpful. Since sunlight is not always available, some benefit may be obtained with a sunlamp.

Another combination therapy involves Azelaic acid cream 20%, which has antiproliferative and antibacterial effects, and is known to be effective in comedonal or inflammatory acne.

An additional combination therapy contemplated with the invention is topical tretinoin (retinoic acid) in 0.025%, 0.05%, or 0.1% cream, 0.05% liquid, or 0.01% or 0.025% gel. Also, a new topical retinoid, Differin® brand adapalene 0.1% gel, Galderma Laboratories, San Antonio, Tex., was recently approved in the USA and may be useful since it may be slightly less irritating than topical tretinoin. Other retinoids which may be useful in combination therapy include Panretin®, containing alitretinoin, and Targretin®, containing bexarotene. Since retinoids must be applied carefully and at night to avoid excessive irritation, a regimen in combination with these drugs may be used over time to achieve results. For example, retinoid therapy may be initiated and then followed on with once a day treatment in accordance with the present invention. Exposure to sunlight when using retinoids and concurrent use of other drugs are restricted to prevent severe irritation. However, a back-to-back alternating regimen over a period of weeks or months time may be useful. With tretinoin or adapalene, acne may worsen at first; improvement usually requires >=3 to 4 weeks.

Other topical drugs include OTC drugs, various sulfur-resorcinol combinations, and oral antibiotics may also be helpful in combination with the present invention when treating superficial pustular acne.

Accordingly a preferred embodiment of the present inventive subject matter additionally relates to a method for the treatment of acne in a patient in need thereof, comprising administering a combination of benzoyl peroxide and clindamycin to said patient, wherein said combination contains a low level of lincomycin phosphate sulfoxide and lincomycin sulfoxide.

### Methods of Use

The present inventive subject matter also relates to a method for treating a skin disorder or condition in a patient comprising topically administering to a patient in need thereof a topical composition in an amount effective to treat said skin disorder, wherein said composition comprises:

a storage-stable mixture of a benzoyl peroxide dispersion, clindamycin or a pharmaceutically acceptable salt or ester thereof, and a pharmaceutically acceptable carrier,

wherein the composition has a final pH of about 4.5 to about 5, and wherein the composition has a viscosity lower than the viscosity of the benzoyl peroxide dispersion before mixing.

Skin disorders or conditions treatable according to the present inventive methods include but are not limited to microbial infections and inflammation of tissue. The microbial infections can be caused by gram-positive bacteria, gram-negative bacteria, and mixtures thereof. Exemplary specific bacteria treatable by the present inventive compositions include but are not limited to P. acnes, Strep. Pyogenes, Staph. Aureus, E. coli, Pseudonomas originosa, and mixtures thereof.

Exemplary specific skin disorders treatable by the present inventive compositions include but are not limited to acne, impetigo, rosacea, atopic dermatitis, secondary skin infections, seborrhea, skin lesions, and bacterial skin infections. In a preferred embodiment, the skin disorder or condition improves following treatment with the present inventive compositions.

In another preferred embodiment, the present inventive subject matter further relates to a method for the treatment of acne in a patient in need thereof, comprising administering a combination of benzoyl peroxide and clindamycin which has been refrigerated to said patient. This combination has a specific degradation profile, in accordance with the data submitted below.

In a preferred embodiment, the patient to be treated is between the ages of 2 and 45. In a particularly preferred embodiment, the patient to be treated is between the ages of 10 and 35. In yet another preferred embodiment, the patient to be treated is between the ages of 12 and 25.

### Process for Preparing

The present inventive subject matter further relates to a process for preparing a storage-stable topical composition for treating for a skin disorder or condition, which comprises the steps of:

a) forming at a temperature of about 15 to about 25 ° C. a benzoyl peroxide intermediate dispersion having between about 5.9% and about 7.2% benzoyl peroxide and having a viscosity of about 60,000 to about 250,000 centipoises;

b) forming at a temperature of about 15 to about 25 ° C. a clindamycin intermediate solution sufficient to yield a composition which contains between about 0.5% and about 1.5% by weight clindamycin active in the final product; and

c) mixing said benzoyl peroxide intermediate dispersion and said clindamycin intermediate solution under conditions sufficient to yield a benzoyl peroxide and clindamycin mixture having final pH of between about 4.5 to about 5.0,

wherein said mixture has a viscosity of about 50,000 to about 200,000 centipoises, and wherein said composition comprises sufficient inactive ingredients to provide storage stability and effectiveness for a treatment period.

The mixture made according to this process preferably comprises a benzoyl peroxide gel intermediate mixed with a clindamycin solution intermediate. The benzoyl peroxide gel intermediate preferably contains between about 5.9% and about 7.2% by weight benzoyl peroxide, most preferably about 6.58% by weight benzoyl peroxide.

Also, the composition is preferably manufactured to have about 1% to about 3% less water by weight as compared to a topical formulation having one of benzoyl peroxide or clindamycin alone, but not both together. Such formulations unexpectedly result in compositions that exhibit less skin sensitivity.

Referring to the formulation process of the present compositions, a gel is initially formed. The gel is composed of a carbomer, disodium monolauryl sulfosuccinate, and disodium EDTA to which methylparaben is added as a preservative. Purified water is used as a diluent.

After the gel is formed, wetting agents and emollients are added. After the pH is adjusted, the active ingredients are added to form the final compound.

As discussed above, the active ingredients can be added to the inert ingredients at the same time or separately.

The resultant combination maintains stability for a minimum of three months at room temperature (e.g. 22° C.) and relative, or ambient, humidity.

### Routes of Administration/Dosage

To be effective, the route of administration for the compositions used in the inventive methods and pharmaceutical compositions must readily affect the target areas. In particular, acne is known to affect the face, neck, back, ears, and scalp.

Dosage levels for the antibiotics and the benzoyl peroxide are well known in the art and are selected to maximize the treatment of the above conditions. The specific dose level for any particular patient will vary depending upon a variety of factors, including the activity of the specific compound employed; the age, body weight, general health, sex and diet of the patient; the time of administration; the rate of excretion; drug combination; the severity of the particular disease being treated; and the form of administration. Typically, in vitro dosage-effect results can provide useful guidance on the proper doses for patient administration. Studies in animal models are also helpful. The considerations for determining the proper dose levels are well known in the art and are incorporated herein for the present inventive subject matter.

Pharmacokinetic parameters such as bioavailability, absorption rate constant, apparent volume of distribution, unbound fraction, total clearance, fraction excreted unchanged, first-pass metabolism, elimination rate constant, half-life, and mean residence time are well known in the art.

Lessening exposure by once-daily administration affects multiple pharmacokinetic parameters and provides the initial mechanism for avoiding skin irritation and inflammation and the other toxicity issues discussed herein. Additional formulations may be prepared which factor in the benefit/risk ratio for a clindamycin and benzoyl peroxide composition. The level of toxicity of these compounds is known and reference is made to the package inserts for Cleocin T® and BenzaClin® and the level of adverse events reported from their clinical trials. In particular, BenzaClin® reported having the following events: dry skin (12%), pruritis (2%), peeling (2%), erythema (1%) and sunburn (1%) as compared to vehicle which reported dry skin (6%), pruritis (<1%), peeling (−), erythema (<1%) and sunburn (−), or roughly twice the number of side effects as vehicle.

Since benzoyl peroxide is a keratolytic, i.e. causes softening and swelling of the cells at the surface of the skin so that the outer layer of the skin peels off or can easily be removed, reducing exposure to it reduces irritation. Upon application, the benzoyl peroxide converts to benzoic acid and has anti-bacterial and anti-fungal properties. Additionally, the low pH of the inventive formulations may have an additive keratolytic effect on the skin as well as on the anti-bacterial properties. Benzoyl peroxide may also acts as a preservative within the formulation. Clindamycin may degrade at pH higher than pH 6, thus requiring the pH to be maintained below this level, as described herein. The formulations of the present invention take these and other factors into account and are manufactured to reduce sensitivity, irritation, and/or inflammation.

Single dosage kits and packages containing once per day amount of composition may be prepared. Single dose, unit dose, and once-daily disposable containers of the mixtures and compositions of the invention are contemplated as within the scope of the inventive subject matter.

The present inventive compositions may be formulated for storage in a substantially non-reactive laminated package to enhance stability of the package. This new method of storage provides enhanced package stability in comparison with the previous paper-based packages.

The amount of composition per single packet may range be from about 0.1 mL to about 20.0 mL, preferably between about 0.5 and about 5.0 mL, more preferably between about 1 and about 3 mL.

In particular, the ability to formulate compositions capable of long term storage, without pre-mixing or compounding requirements prior to application, are also contemplated. Specifically, the compositions of the present invention remain unexpectedly stable in storage for periods including between about 3 and about 18 months, preferably between about 3 and about 15 months, more preferably between about 3 and about 12 months, and alternately any time period between about 6 and about 18 months. In this regard, while the product may be refrigerated during the distribution and pharmacy storage phases, the product does not need refrigeration for the about 3 months and longer as stated above when stored by the patient at room temperature.

Once-daily disposable packaging may also improve patient compliance, especially for teenagers.

The stability and effectiveness of the topical preparations may last for at least 3 to 18 months at ambient or room temperature. It has been found that the greater the amount of clindamycin in the final product, the greater the stability is maintained. Stability is maintained indefinitely under refrigeration because degradation is slowed through the storage temperature. This improved stability provides pharmacists and other dispensers of medication with a product which no longer requires compounding at the time of dispensing. Because compounding is no longer required, homogeneity is controlled at the point of manufacture, which improves dosing and ultimately compliance. Furthermore, the present invention does not employ alcohol as a diluent, which eliminates the drying or irritating effects commonly associated therewith.

Stability of the composition is maintained for longer periods of time depending on the amount of clindamycin employed in the final product and the ratio of benzoyl peroxide to clindamycin. For example, when 1.2% of clindamycin is present in the compound, the shelf life can reach from seven to fourteen months at room temperature while maintaining effectiveness. In contrast, when only 1.02% of clindamycin is employed, the shelf life of the product is closer to three months.

Differences in packaging components and manufacturing techniques yield varied formula responses over a period ranging between seven and fourteen months in stability testing as evidenced by the following data:

| Ref No. | BPO/Clindamycin Ratio | Minimum Projected Stability |
|---|---|---|
| A | 5/1.2 | 14 months |
| B | 5/1.2 | 9 months |
| C | 5/1.2 | 7 months |
| D | 5.9/1 | 7 months |
| E | 5/1.02 | 3 months |
| F | 5/1.02 | 3 months |

In addition to the amount of clindamycin as a control over degradation, the temperature at which the composition is stored determines the length of time that the composition remains stable. When the composition is stored at a temperature below ambient temperature (25° C.), the stability is maintained indefinitely. For example, storing the compound at 6° C. with the proper amount of overage of clindamycin results in an anticipated shelf life of 3 to 5 years.

Advantageously, the final product requires no compounding by the pharmacist. In addition, compliance with exact amounts is possible with a lessened chance of impurities entering the product and contaminating it.

By maintaining the compositions at the present specific pH, the tendency of benzoyl peroxide to oxidize and degrade clindamycin is largely overcome and the product remains stable during storage at room temperature for extended periods, typically several months or longer. Additionally, the compositions of the present invention have been found to remain substantially odor free even after storage at room temperature for extended periods. This is surprising since clindamycin solutions frequently develop a strong offensive odor upon aging. The presence of such an odor is unacceptable in topical formulations which are to be applied to a patient's face.

The following examples are illustrative of preferred embodiments of the invention and are not to be construed as limiting the invention thereto. All polymer molecular weights are mean average molecular weights. All percentages are based on the percent by weight of the final delivery system or formulation prepared unless otherwise indicated and all totals equal 100% by weight.

In the examples, the following ingredients are used: carbomers and polymeric emulsifiers (polyacrylates), such as for example Carbopol® 940 from Noveon Inc., Cleveland, Ohio; disodium monolauryl sulfosuccinate, such as Monamate LA-100; emulsifier-solubilizer-stabilizers block PEG/PPG co-polymers such as Poloxamer 182, also known as Pluracare® L-62; surfactant-emollient-lubricant-plasticizers such as dimethicone also known as Dow Fluid 200; sequestering agents such as disodium EDTA, commercially known as Hampene® Na₂; and hydrated silica and absorbents, such as Syloid 244 FP.

### EXAMPLE 1

A highly stable gel composition is prepared using the following components. The active ingredients are benzoyl peroxide and clindamycin phosphate. The remaining components are inert or auxiliary.

| Ingredient | Parts by Weight. |
|---|---|
| Gel: | |
| Purified Water | 86.50% |
| Carbomer | 2.00% |
| Disodium monolauryl | 0.04% |
| sulfosuccinate | |
| Disodium EDTA | 0.10% |
| Methylparaben | 0.30% |
| Total: | 88.94% |

The gel is combined with the following to produce the instant composition:

| Wetting Agents and Emollients: | |
|---|---|
| Poloxamer 182 | 0.20% |
| Glycerin | 4.00% |
| Dimethicone | 0.10% |
| Hydrated Silica | 0.25% |
| Total: | 4.55% |
| pH Adjustment: | |
| Sodium Hydroxide | 0.31% |
| Total: | 0.31% |
| Active Ingredients: | |
| Benzoyl Peroxide | 5.00% |
| Clindamycin Phosphate | 1.20% |
| Total: | 6.20% |
| Total for Composition: | 100.00% |

### EXAMPLE 2

The following composition is obtained when the following component formulations are mixed in equal parts, and later combined to yield the highly stable product.

| Ingredient | Parts by Weight |
|---|---|
| Gel: | |
| Purified Water | 82.70% |
| Carbomer | 2.00% |
| Disodium monolauryl | 0.04% |
| sulfosuccinate | |
| Disodium EDTA | 0.10% |
| Methylparaben | 0.12% |
| Total: | 85.14% |

The gel is combined with the following to produce the instant composition:

| Wetting Agents and Emollients: | |
|---|---|
| Poloxamer 182 | 0.20% |
| Glycerin | 4.00% |
| Dimethicone | 0.10% |
| Hydrated silica | 0.25% |
| Total: | 4.55% |
| pH Adjustment: | |
| Sodium Hydroxide | 0.31% |
| Total: | 0.31% |
| Active Ingredients: | |
| Benzoyl Peroxide | 10.00% |
| Clindamycin Phosphate | — |
| Total: | 10.00% |
| Total for Composition: | 100.00% |

| Ingredient | Parts by Weight |
|---|---|
| Gel: | |
| Purified Water | 90.30% |
| Carbomer | 2.00% |
| Disodium monolauryl | 0.04% |
| sulfosuccinate | |
| Disodium EDTA | 0.10% |
| Methylparaben | 0.30% |
| Total: | 92.74% |

The gel is combined with the following to produce the instant composition:

| Wetting Agents and Emollients: | |
|---|---|
| Poloxamer 182 | 0.20% |
| Glycerin | 4.00% |
| Dimethicone | 0.10% |
| Hydrated silica | 0.25% |
| Total: | 4.55% |
| pH Adjustment: | |
| Sodium Hydroxide | 0.31% |
| Total: | 0.31% |
| Active Ingredients: | |
| Benzoyl Peroxide | — |
| Clindamycin Phosphate | 2.40% |
| Total: | 2.40% |
| Total for Composition: | 100.00% |

The resultant mixture is essentially 10% of benzoyl peroxide with essentially 2% clindamycin.

### EXAMPLE 3

Tables 1 and 2 show the stability of the active ingredients. A fourteen-month analysis was performed on a 5.9% benzoyl peroxide and 1% clindamycin gel formulation. Measurements were taken at the end of 3 months and every month thereafter until the 8th month. No measurements were taken at 8 months. Thereafter, measurements were taken at 9, 12, and 14 months. The composition was stored at 3 different temperatures, i.e., 6° C., 25° C., and 30° C. The level of clindamycin was measured at each temperature, as well as the amount of benzoyl peroxide. The results are as follows:

TABLE 1
Benzoyl Peroxide 5% (5.9% in formula)
and clindamycin 1% (1% in formula)
Clindamycin (as % w/w)

| | 6° C. | 25° C. | 30° C. |
|---|---|---|---|
| Initial | | 1.01 | |
| 3 months | 0.95 | 0.90 | 0.77 |
| 4 months | 1.01 | 0.95 | 0.79 |
| 5 months | 1.04 | 0.95 | 0.79 |
| 6 months | 0.96 | 0.91 | 0.71 |
| 7 months | 1.05 | 0.92 | 0.70 |
| 9 months | 1.03 | ND | ND |
| 12 months | 0.93 | 0.79 | 0.37 |
| 14 months | 0.93 | 0.76 | 0.27 |

ND = No Data

TABLE 2
Benzoyl Peroxide (BPO) (as % w/w)

| | 6° C. | 25° C. | 30° C. |
|---|---|---|---|
| Initial | | 6.13 | |
| 3 months | 5.97 | 5.90 | 5.98 |
| 4 months | 6.07 | 6.05 | 5.98 |
| 5 months | 6.08 | 5.96 | 5.84 |
| 6 months | 6.13 | 6.04 | 5.91 |
| 7 months | 6.23 | 6.19 | 6.06 |
| 9 months | 6.02 | 5.95 | |
| 12 months | 5.95 | 5.89 | 5.63 |
| 14 months | 6.10 | 6.10 | 5.77 |

### EXAMPLE 4

Tables 3 and 4 show the stability of the active ingredients in the composition containing 5% of benzoyl peroxide and 1.2% of clindamycin.

A six-month analysis of the composition was undertaken following the procedure of Example 3 and utilizing a different amount of clindamycin and benzoyl peroxide.

TABLE 3
Benzoyl Peroxide 5% (BPO) (5.9% in formula)
and clindamycin 1% (1.2% in formula)
Clindamycin (as % w/w)

| | 6° C. | 25° C. | 30° C. |
|---|---|---|---|
| Initial | | 1.24 | |
| 1 months | 1.25 | 1.24 | 1.15 |
| 2 months | 1.28 | 1.21 | 1.01 |
| 3 months | 1.23 | 1.13 | 0.94 |
| 6 months | 1.21 | 1.05 | ND |

TABLE 4
Benzoyl Peroxide (BPO) (as % w/w)

| | 6° C. | 25° C. | 30° C. |
|---|---|---|---|
| Initial | | 5.09 | |
| 1 months | 5.10 | 5.02 | 5.08 |
| 2 months | 5.25 | 5.20 | 5.13 |
| 3 months | 5.16 | 5.18 | 4.82 |
| 6 months | 5.07 | 5.06 | ND |

ND = No Data

### EXAMPLE 5

Tables 5 through 13 show the stability of the active ingredients. An analysis of at least 24 months was performed following the procedure of Example 3. Measurements were taken after storage for a specified number of months at 6° C. followed by storage for 91 days thereafter at 25° C.

The columns in the following tables represent the following components (as % w/w):

Column A—Clindamycin

Column B—Clindamycin HCl

Column C—Clindamycin B-2 Phosphate

Column D—Clindamycin Phosphate Sulfoxide Isomer 1 (Clindamycin Degradate 1)

Column E—Clindamycin Phosphate Sulfoxide Isomer 2 (Clindamycin Degradate 2)

Column F—Lincomycin Phosphate Sulfoxide (Clindamycin Degradate 3)

TABLE 5
Benzoyl Peroxide 5% (BPO)
and clindamycin 1%
Trial 1

| | | | | | | | pH | Package |
|---|---|---|---|---|---|---|---|---|
| | A | B | C | D | E | F | Range | Size |
| Initial | 1.01 | <0.005 | 0.012 | 0.037 | 0.047 | ND | 4.6-4.7 | 5 g |
| 25 months | 0.95 | <0.005 | 0.008 | 0.041 | 0.074 | 0.007 | 4.6-4.7 | 5 g |
| 30 months | 0.93 | <0.005 | 0.007 | 0.044 | 0.082 | 0.003 | 4.6-4.7 | 5 g |
| 36 months | 0.93 | <0.005 | <0.001 | 0.050 | 0.086 | 0.003 | 4.6-4.7 | 5 g |
| 48 months | 0.89 | <0.005 | <0.001 | 0.062 | 0.111 | 0.003 | 4.6-4.8 | 5 g |

TABLE 6
Benzoyl Peroxide 5% (BPO)
and clindamycin 1%
Trial 2

| | | | | | | | pH | Package |
|---|---|---|---|---|---|---|---|---|
| | A | B | C | D | E | F | Range | Size |
| Initial | 1.02 | <0.005 | 0.013 | 0.011 | 0.016 | ND | 4.6-4.7 | 45 g |
| 21 months | 0.96 | <0.005 | <0.001 | 0.046 | 0.070 | 0.008 | 4.6-4.8 | 45 g |
| 24 months | 0.95 | <0.005 | 0.008 | 0.048 | 0.084 | 0.008 | 4.6-4.7 | 45 g |
| 30 months | 0.93 | <0.005 | 0.007 | 0.045 | 0.083 | 0.003 | 4.6-4.7 | 45 g |
| 36 months | 0.93 | <0.005 | <0.001 | 0.048 | 0.086 | 0.003 | 4.6-4.8 | 45 g |

TABLE 7
Benzoyl Peroxide 5% (BPO)
and clindamycin 1%
Trial 3

| | | | | | | | pH | Package |
|---|---|---|---|---|---|---|---|---|
| | A | B | C | D | E | F | Range | Size |
| Initial | 1.05 | <0.005 | 0.008 | <0.001 | 0.005 | ND | 4.8 | 45 g |
| 24 months | 0.92 | <0.005 | <0.001 | 0.044 | 0.079 | 0.003 | 4.6-4.8 | 45 g |
| 30 months | 0.93 | <0.005 | <0.001 | 0.048 | 0.087 | 0.002 | 4.7-4.8 | 45 g |
| 37 months | 0.91 | <0.005 | <0.001 | 0.055 | 0.095 | 0.003 | 4.5-4.9 | 45 g |
| 42 months | 0.89 | <0.005 | <0.001 | 0.058 | 0.106 | 0.002 | 4.7-4.8 | 45 g |
| 48 months | 0.89 | <0.005 | <0.001 | 0.058 | 0.110 | 0.003 | 4.6-4.7 | 45 g |

TABLE 8
Benzoyl Peroxide 5% (BPO)
and clindamycin 1%
Trial 4

| | | | | | | | pH | Package |
|---|---|---|---|---|---|---|---|---|
| | A | B | C | D | E | F | Range | Size |
| 24 months | 0.92 | <0.005 | <0.001 | 0.044 | 0.080 | 0.004 | 4.7-4.8 | 45 g |
| 30 months | 0.91 | <0.005 | <0.001 | 0.044 | 0.075 | 0.002 | 4.7-4.8 | 45 g |
| 37 months | 0.90 | <0.005 | <0.001 | 0.049 | 0.090 | 0.002 | 4.7-4.8 | 4.5 g |
| 42 months | 0.89 | <0.005 | <0.001 | 0.058 | 0.103 | 0.002 | 4.7-4.8 | 45 g |

TABLE 9
Benzoyl Peroxide 5% (BPO)
and clindamycin 1%
Trial 5

| | | | | | | | pH | Package |
|---|---|---|---|---|---|---|---|---|
| | A | B | C | D | E | F | Range | Size |
| Initial | 1.01 | <0.005 | 0.008 | <0.001 | 0.005 | ND | 4.7-4.9 | 5 g |
| 24 months | 0.91 | <0.005 | <0.001 | 0.044 | 0.070 | 0.004 | 4.6-4.8 | 5 g |
| 30 months | 0.90 | <0.005 | <0.001 | 0.040 | 0.073 | 0.002 | 4.7-4.8 | 5 g |
| 37 months | 0.88 | <0.005 | <0.001 | 0.052 | 0.093 | 0.003 | 4.4-4.8 | 5 g |
| 42 months | 0.89 | <0.005 | <0.001 | 0.057 | 0.104 | 0.002 | 4.7-4.8 | 5 g |

TABLE 10
Benzoyl Peroxide 5% (BPO)
and clindamycin 1%
Trial 6

| | | | | | | | pH | Package |
|---|---|---|---|---|---|---|---|---|
| | A | B | C | D | E | F | Range | Size |
| 24 months | 0.91 | <0.005 | <0.001 | 0.046 | 0.075 | 0.001 | 4.7-4.8 | 45 g |
| 31 months | 0.90 | <0.005 | <0.001 | 0.042 | 0.080 | 0.001 | 4.7-4.8 | 45 g |
| 36 months | 0.88 | <0.005 | <0.001 | 0.055 | 0.101 | 0.001 | 4.7-4.0 | 45 g |

TABLE 11
Benzoyl Peroxide 5% (BPO)
and clindamycin 1%
Trial 7

| | | | | | | | pH | Package |
|---|---|---|---|---|---|---|---|---|
| | A | B | C | D | E | F | Range | Size |
| Initial | 1.01 | <0.005 | <0.001 | 0.005 | 0.008 | 0.005 | 4.7 | 45 g |
| 24 months | 0.91 | <0.005 | <0.001 | 0.045 | 0.079 | <0.001 | 4.7-4.8 | 45 g |

TABLE 12
Benzoyl Peroxide 5% (BPO)
and clindamycin 1%
Trial 8

| | | | | | | | pH | Package |
|---|---|---|---|---|---|---|---|---|
| | A | B | C | D | E | F | Range | Size |
| Initial | 1.03 | <0.005 | <0.001 | 0.006 | 0.010 | 0.002 | 4.7 | 45 g |

TABLE 13
Benzoyl Peroxide 5% (BPO)
and clindamycin 1%
Trial 9

| | | | | | | | pH | Package |
|---|---|---|---|---|---|---|---|---|
| | A | B | C | D | E | F | Range | Size |
| Initial | 1.04 | <0.005 | <0.001 | 0.006 | 0.009 | 0.002 | 4.7-4.8 | 45 g |

The invention being thus described, it will be obvious that the same may be varied in many ways. Such variations are not to be regarded as a departure from the spirit and scope of the invention and all such modifications are intended to be included within the scope of the following claims.

We claim:

1. A topical composition for treating a skin disorder or condition, which comprises:
a storage-stable mixture of a benzoyl peroxide dispersion, clindamycin or a pharmaceutically acceptable salt or ester thereof, and a pharmaceutically acceptable carrier,
wherein the composition has a final pH of about 4.5 to about 5, and wherein the composition has a viscosity lower than the viscosity of the benzoyl peroxide dispersion before mixing.

2. The composition of claim 1, wherein said composition is formulated for once-per-day administration.

3. The composition of claim 2, wherein said once-per-day administration occurs in the A.M.

4. The composition of claim 1, wherein said composition is formulated for twice-per-day administration.

5. The composition of claim 1, wherein said composition has a final pH of about 4.6 to about 4.8.

6. The composition of claim 1, wherein said dispersion is selected from the group consisting of a gel, cream, lotion, suspension, emulsion, ointment, foam, and mixtures thereof.

7. The composition of claim 1, wherein said composition is stored at a temperature of less than about 30° C.

8. The composition of claim 1, wherein said composition is storage-stable for commercial purposes.

9. The composition of claim 1, wherein said composition has a final viscosity of about 50,000 to about 200,000 centipoises.

10. The composition of claim 9, wherein said composition has a final viscosity of about 100,000 to about 200,000 centipoises.

11. The composition of claim 1, wherein said benzoyl peroxide dispersion has a viscosity of about 60,000 to about 250,000 centipoises.

12. The composition of claim 11, wherein said benzoyl peroxide dispersion has a viscosity of about 110,000 to about 220,000 centipoises.

13. The composition of claim 1, wherein said benzoyl peroxide is about 65% to about 80% pure.

14. The composition of claim 1, wherein said mixture comprises about 1% to about 20% by weight percent benzoyl peroxide.

15. The composition of claim 14, wherein said mixture comprises about 2.25% to about 12.5% by weight benzoyl peroxide.

16. The composition of claim 1, wherein said composition further contains inactive ingredients selected from the group consisting of carbomer, disodium monolauryl sulfosuccinate, disodium EDTA, methyl paraben, poloxamer, glycerin, dimethicone, hydrated silica, sodium hydroxide, purified water, and mixtures thereof.

17. A method for treating a skin disorder or condition in a patient comprising topically administering to a patient in need thereof a topical composition in an amount effective to treat said skin disorder, wherein said composition comprises:
a storage-stable mixture of a benzoyl peroxide dispersion, clindamycin or a pharmaceutically acceptable salt or ester thereof, and a pharmaceutically acceptable carrier,
wherein the composition has a final pH of about 4.5 to about 5, and wherein the composition has a viscosity lower than the viscosity of the benzoyl peroxide dispersion before mixing.

18. The method of claim 17, wherein said skin disorder or condition includes a microbial infection.

19. The method of claim 18, wherein said microbial infection is caused by bacteria selected from the group consisting of gram-positive bacteria, gram-negative bacteria, and mixtures thereof.

20. The method of claim 19, wherein said bacteria is selected from the group consisting of P. acnes, Strep. Pyogenes, E. coli, Pseudonomas originosa, Staph. Aureus, and mixtures thereof.

21. The method of claim 17, wherein said skin disorder or condition includes an inflammation of tissue.

22. The method of claim 21, wherein said skin disorder is selected from the group consisting of acne, impetigo, rosacea, atopic dermatitis, secondary skin infections, and mixtures thereof.

23. The method of claim 17, wherein said patient is between the ages of 2 and 45.

24. The method of claim 23, wherein said patient is between the ages of 10 and 35.

25. The method of claim 24, wherein said patient is between the ages of 12 and 25.

26. A process for preparing a storage-stable topical composition for treating for a skin disorder or condition, which comprises the steps of:
a) forming at a temperature of about 15 to about 25° C. a benzoyl peroxide intermediate dispersion having between about 5.9% and about 7.2% benzoyl peroxide and having a viscosity of about 60,000 to about 250,000 centipoises;
b) forming at a temperature of about 15 to about 25 ° C. a clindamycin intermediate solution sufficient to yield a composition which contains between about 0.5% and about 1.5% by weight clindamycin active in the final product; and
c) mixing said benzoyl peroxide intermediate dispersion and said clindamycin intermediate solution under conditions sufficient to yield a benzoyl peroxide and clindamycin mixture having final pH of between about 4.5 to about 5.0,
wherein said mixture has a viscosity of about 50,000 to about 200,000 centiposies, and wherein said composition comprises sufficient inactive ingredients to provide storage stability and effectiveness for a treatment period.

27. The process of claim 26, wherein said process results in a composition having benzoyl peroxide impurities of not more than about 0.01% by weight.

28. The process of claim 26, wherein said process results in a composition having clindamycin degradates of not more than about 0.02% by weight.

29. The process of claim 26, wherein said process results in a composition having benzoyl peroxide impurities of not more than about 0.01% by weight and clindamycin degradates of not more than about 0.02% by weight.

30. The process of claim 26, wherein said mixture has a final pH of between about 4.6 to about 4.8.

31. The process of claim 26, wherein said composition has less water by weight as compared to a topical formulation having one of benzoyl peroxide or clindamycin but not both.

32. A dermatological composition for topical treatment of skin disorders, which comprises:
a composition produced according to the process of claim 26, having a standard deviation of amount of benzoyl peroxide present within ±0.07 and a standard deviation of amount of clindamycin present within ±0.015.

33. The composition of claim 32, wherein said composition is selected from the group consisting of a gel, cream, lotion, suspension, emulsion, ointment, foam, and mixtures thereof.

34. The composition of claim 33, wherein said composition is a gel.

35. The composition of claim 1, wherein said composition is stored in a substantially non-reactive laminated package to enhance stability of the package.
